(19) United States
(12) Patent Application Publication
(10) Pub. No.: US 2008/0249073 A1
(43) Pub. Date: Oct. 9, 2008

(54) Skin Treatment Composition

(75) Inventors: Mike Farwick, Essen (DE); Anthony V. Rawlings, Chesshire (GB)

Correspondence Address: SCULLY SCOTT MURPHY & PRESSER, PC, 400 GARDEN CITY PLAZA, SUITE 300, GARDEN CITY, NY, 11530, US

(73) Assignee: EVONIK GOLDSCHMIDT GMBH, Essen (DE)

(21) Appl. No.: 12/135,462

(22) Filed: Jun. 9, 2008

Related U.S. Application Data
(63) Continuation of application No. 11/479,237, filed on Jun. 30, 2006.

(30) Foreign Application Priority Data
Jun. 30, 2005 (EP) 05014134.0

(51) Int. Cl. A61K 31/606; A61P 17/00

(52) U.S. Cl. 514/166

(57) ABSTRACT

This invention relates to a cosmetic composition comprising:
a) Salicyloyl-sphingoidbases and/or derivatives thereof and
b) a dermatologically acceptable carrier
   for treating and/or preventing cellulitic skin by increasing dermal structural proteins (collagen and fibrillin) and reducing the levels of MMP-1 activity.

### CROSS REFERENCE TO RELATED APPLICATIONS

The present application is a contination of copending application Ser. No. 11/479,237 filed on Jun. 30,2006.

### FIELD OF THE INVENTION

This invention relates to topical compositions containing salicyloyl-sphingoid bases for application to human skin for the treatment and/or prevention of cellulite.

### BACKGROUND AND PRIOR ART

Skin is subject to deterioration through the passage of time (chronological aging, dermatological disorders, hormonal changes, environmental abuse (wind, air conditioning, central heating, pollution, etc.) all of which may be accelerated by exposure of skin to radiation from the sun (photoaging). Consumers are increasingly seeking “anti-aging” cosmetic products which treat, or delay, the visible signs of chronological aging and photo-aging skin such as wrinkles, lines, sagging, hyperpigmentation and age spots. Cellulite also manifests itself as a result of increased adipogenicity and reduced connective tissue matrix.

Skin care cosmetic and dermatological compositions for improving the condition and appearance of skin comprising sphingolipids operate mainly in the epidermal layers of the skin.

There continues to be a need, however, for alternative effective cosmetic compositions for topical application to skin for treating/delaying the visible signs of aging and photo-damaged skin such as wrinkles, lines, sagging, hyper-pigmentation, age spots and especially cellulite which are more dermal effects.

### SUMMARY OF THE INVENTION

Sphingolipid derivatives and ceramides have been previously used to treat the barrier of the skin (i.e., the epidermis). It has now been surprisingly found that effective treatment and prevention of skin conditions due to chronoaging or photoaging, such as wrinkles, lines, sagging, hyperpigmentation and age spots, and/or of celluliter may be obtained through the application of cosmetic compositions to the skin which comprise a specific sphingolipid: salicyloyl-phytosphingosine, acting primarily to increase dermal and dermoepidermal junctional proteins.

According to a first aspect of the present invention, there is provided a topical composition comprising:
(a) at least one sahcyloyl-phytosphingosine or derivative thereof; and
(b) a dermatologically acceptable carrier.

Such compositions are particularly useful for topical application to human skin for cosmetically treating/preventing skin conditions selected from the group consisting of wrinkling, sagging and, especially, cellulitic skin. The compositions of the present application are also useful for application to the skin as a cosmetic treatment, which promotes dermal repair and boosts collagen and fibrillin deposition in skin.

The inventive compositions and treatment methods thus provide anti-aging and anti-cellulite benefits which result in the promotion of smooth and supple skin with, improved elasticity and a reduced or delayed appearance of wrinkles, aged and/or orange-peel skin. A general improvement in the appearance, texture and condition, in particular with respect to the radiance, clarity, firmness, smoothness and general youthful appearance of skin is achieved. Thus, the inventive treatment methods and compositions advantageously provide a wide range of skin care benefits.

The term “treating” as used herein includes within its scope reducing, delaying and/or preventing the appearance of wrinkled, aged, photodamaged, dry, cellulitic and generally enhancing the quality of skin and improving its appearance and texture by preventing or reducing wrinkling and increasing flexibility, firmness, smoothness, suppleness and elasticity of the skin. The cosmetic compositions, methods and the uses of the salicyloyl-phytosphingosine according to the invention may be useful for treating skin which is already in a wrinkled, aged, photodamaged, dry and, especially, cellulitic condition, or for treating youthful skin to prevent or reduce those aforementioned deteriorative changes due to the normal aging/photo aging process.

### DETAILED DESCRIPTION OF THE INVENTION

Details of structure and/or derivatives thereof.

Phytosphingosine has the structure shown below:

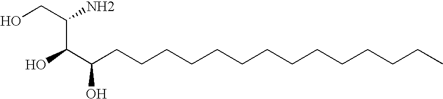

A particular derivative of phytosphingosine is salicyloyl-phytosphingosine. It has the structure shown below:

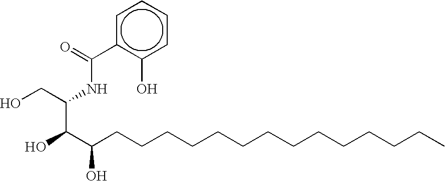

The invention also includes other derivatives. Preferable derivatives include derivatives of the hydroxyl groups of the phytosphingosine (PS) moiety, such as organic and inorganic esters, e.g., phosphate esters, sulphate (i.e., sulfate) esters and acetate esters or glycosylated derivatives. The invention also includes other salicyloyl-sphingoidbase derivatives, e.g., salicyloyl-sphingosine, salicyloyl-sphinganine, salicyloyl-6-hydroxy-sphinganine.

A preferred method of preparation is disclosed in Example 1 below.

The active, salicyloyl-phytosphingosine, to be employed in accordance with the present invention is present in the topical composition in an effective amount Normally the total amount of the active is present in an amount between 0.001 and 20% by weight of the composition. More preferably, the amount is from 0.01 to 10% and most preferably from 0.02 to 2% in order to maximize benefits at a minimum cost.

### Optional Skin Benefit Materials and Cosmetic Adjuncts

Besides the active, salicyloyl-phytosphingosine, other specific skin-benefit actives such as sunscreens, skin lightening agents, skin tanning agents may also be included. The vehicle may also further include adjuncts such as perfumes, antioxidants, opacifiers, preservatives, colourants and buffers.

Typical additional bioactive compounds are tocopherol and derivatives, ascorbic acid and derivatives, desoxyribonucleic acid, retinol and derivatives, caffeine and derivatives, alpha-lipoic acid, bisabolol, allantoin, phytantriol, panthenol, alpha hydroxy acids, amino acids, hyaluronic acid and derivatives, creatine and derivatives, trimethylglycine, myoinositol, pyroglutamatic acid and derivatives, taurine, guanidine and derivatives, phospholipids, lysophospholipids, ceramides, phytosphingosine and derivatives, sphingosine and derivatives, pseucfoceramides and derivatives, essential oils, peptides, modified peptides, protein hydrolysates, plant extracts and vitamin complexes.

As well as in skin care applications as described above, salicyloyl-phytosphingosine can also be incorporated into hair care formulations such as, for example, shampoos and conditioners, where it has a stimulating effect on the scalp performance.

### Dermatologically Acceptable Vehicle

The composition according to the invention also comprises a dermatologically/cosmetically acceptable vehicle to act as a diluent, dispersant or carrier for the active, salicyoyl-phytosphingosine. The vehicle may comprise materials commonly employed in skin care products such as water, liquid or solid emollients, silicone oils, emulsifiers, solvents, humectants, thickeners, powders, propellants and the like. Other agents which can be employed in the present application as the dermatologically acceptable vehicle include, for example:

Sphingoid and phospholipid derivatives: ceramides, phytosphingosine, sphingosine, pseudoceramides, phospholipids, lysophospholipids.

Antioxidants and vitamins: tocopherol and derivatives, ascorbic acid and derivatives, niacinamide and derivatives, vitamin complexes, alpha-lipoic acid, retinol and derivatives, panthenol.

Antiinflammatories: bisabolol, allantoin, phytantriol, Coenzyme Q10, Idebenone.

Botanical agents such as polyphenolics, flavonoids or isoflavones.

Moisturising agents: amino acids, hyaluronic acid and derivatives, creatine and derivatives, trimethylglycine, myoinositol, pyroglutamatic acid and derivatives, taurine, guanidine and derivatives and hydroxy acids.

Skin whitening agents: kojic acid, arbutin, vitamin C and derivatives, hydroquinone.

Peptides, modified peptides, protein hydrolysates.

Caffeine.

Sunscreens and UV-Absorbers.

The dermatologically acceptable vehicle will usually form from 80 to 99.99%, preferably from 90 to 99.99% and most preferably from 98 to 99.98% by weight of the composition, and can, in the absence of other cosmetic adjuncts, form the balance of the composition.

The skin care formulation can be an aqueous solution, a water-in-oil (w/o) emulsion, an oil-in-water (o/w) emulsion, a dispersion of lipids, an aqueous, water-alcohol, oil or oil-alcohol gel, a solid stick, a wet-wipe or an aerosol.

If the dermatologically acceptable vehicle itself is an (w/o) or (o/w) emulsion, it can contain 5 to 50% of an oilphase and 47 to 94.95% water, with respect to the weight of the whole formulation.

Typical guideline formulations are public available and can be obtained by the manufacturers of either the raw materials or the active ingredients (e.g.,: Kosmetik-Jahrbuch, B. Ziolkowsky, Verlag für Chemische Industrie, Germany). The cosmetic formulations of the present invention show an anti-ageing, anti-wrinkle and/or an anti-cellulite effect

### Product Preparation, Form, Use and Packaging

To prepare the topical composition according to the present invention, the usual manner for preparing skin care products may be employed. The active components are generally incorporated in a dermatologically acceptable carrier in conventional manner. The active components can suitably be dissolved or dispersed in a portion of the water or another solvent or liquid to be incorporated in the composition. The preferred compositions are oil-in-water or water-in-oil emulsions.

The composition may be in the form of conventional skin-care products such as a cream, gel or lotion or the like. The composition can also be in the form of a so-called “rinse-off” product, e.g., a bath or shower gel, possibly containing a delivery system for the actives to promote adherence to the skin during rinsing. Most preferably, the product is a “leave-on” product; a product to be applied to the skin without a deliberate rinsing step soon after its application to the skin.

The inventive composition may be packaged in any suitable manner such as in a jar, a bottle, tube, roll-ball, or the like, in the conventional manner.

The active ingredients described in the present invention maybe applied one or more times daily to the skin, which requires treatment The improvement in skin appearance will usually become visible after 1 to 6 months, depending on skin condition, the concentration of the active components used in the inventive method, the amount of composition used and the frequency with which it is applied, hi general, a small quantity of the composition, for example from 0.1 to 5 ml is applied to the skin from a suitable container or applicator and spread over and/or rubbed into the skin using the hands or fingers or a suitable device. A rinsing step may optionally follow depending on whether the composition is formulated as a “leave-on” or a “rinse-off” product

In order that the present invention may be more readily understood, the following examples are given, by way of illustration only.

Collagen, the predominant matrix skin protein is known to impart tensile strength to skin. Fibrillin is an important protein acting at the dermoepidermal junction of the skin. It is also known in the art that the levels of collagen and fibrillin in skin are significantly reduced with aged and/or photodamaged skin. Many studies have shown that the levels of collagen type I in skin is decreased with age and/or with increased photodamage, (for example: Lavker, R. J. Inv. Derm.(1979), 73,79-66; Griffiths et al. N. Eng. J. med. (1993), 329,530-535). In the case of fibrillin, it has been shown by Watson et al that its expression is greatly reduced in photodamaged skin (Watson R E B, Griffiths C E M, Craven N M, Shuttleworth C A, Kielty C M, Fibrillin-rich microfibrils are reduced in photoaged skin: Distribution at the dermo-epidermal junction, J Invest Dermatol, 112: 782-787,1999). The reduction of the levels of these skin proteins is accordingly associated with a decrease in the tensile strength of the skin causing wrinkles and laxity. As a result of this laxity cellulite is also more visible.

It is well known in the art that retinoic acid is a potent anti-aging active and induces dermal repair of photodamaged skin. It has been shown that wrinkle effacement and dermal repair following topical treatment of skin with retinoic acid arises through new collagen deposition and synthesis in the skin (for example: Griffiths et al, N. Eng. J. med (1993), 329,530-535). It is widely accepted that strengthening of the dermal matrix by boosting the level of collagen in skin using retinoic acid provides anti-aging/dermal repair benefits. Procollagen I is a precursor of collagen. Increased production of procollagen I in response to a test compound application is a marker of an increased collagen level. Matrix-Metallo-Proteases (MMP) are collagen degrading enzymes. Their decreased expression will lead to less collagen degradation and thus to an increased collagen level. Fibrillin located at the Dermal-Epidermal-Junction (DEJ) anchors the collagen fibres within the DEJ and leads to a improved connection between dermis and epidermis. Its level is decreased in photodamaged skin and an increase if the fibrillin levels provide anti-aging/dermal repair benefits.

### EXAMPLES

### Example 1

This example demonstrates the anti-aging benefits of a salicyoyl-phytosphingosine (PS-SLC) and phytosphingosine (PS).

It was done by identification of Procollagen-I and Fibrillin upregulation and matrix metalloprotease-1 (MMP-1) down-regulation in Skin In Vivo. Experiments were done following topical Retinoic Acid (RA) treatment for comparative purposes and analysis of the effects of salicyloyl-phytosphingosine and phytosphingosine compared with retinoic acid with respect to the expression of dermal markers in vivo. See: Watson R E B, Craven N M, Kang S, Jones C J P, Kielty C M, Griffiths C E M. A short term screening protocol, using fibrillin-1 as a receptor molecule for photoageing repair agents. J Invest Dermatol, 116:672-678,2001, for published methodology.

Patch-test protocol and quantification of markers

Five healthy, but clinically photoaged volunteers, were recruited (age range 54-71 years). Test substances were applied separately under standard 6 mm diameter Finn chambers to the extensor aspect of the forearm: these were vehicle base (15 μl/chamber), 0.2% PS-SLC (15 μl/chamber), 0.05% PS-SLC (15 μl/chamber), 0.2% phytosphingosine (15 μl/chamber) and 0.025% all-trans RA (Retin-A® cream, Janssen-Cilag Ltd., 15 μl/chamber). Formulations were applied to clean skin on days 1 and 4 of the assay. All-trans RA was applied to an untreated site on day 4. On day 8, Finn chambers were removed and 3 mm punch biopsies were obtained under 1% lignocaine anaesthesia from each of the test site. Biopsies were embedded in OCT compound (Tissue-Tek®, Miles, Ind., USA) and snap frozen in liquid nitrogen. Biopsy sites were sutured with 1×4/o ethilon and subjects instructed to return between 7-10 days for suture removal. Frozen sections were prepared at a thickness of 10 μm (OTF cryostat, Bright Instruments Ltd.) and mounted onto gelatin-coated slides prior to histological analysis. A number of extracellular matrix (ECM) molecules known to be altered in photoaged skin were assayed by immunohistochemistry to detail the potential effects of the novel formulations. The primary marker of outcome was the distribution of fibrillin-rich microfibrils proximal to the dermal-epidermal junction (DEJ). Also assessed was the distribution of procollagen-1 within the dermis.

Matrix metalloprotease-1 (MMP-1) levels were also assessed. Whilst the fibrillin and procollagen-1 should increase for an antiaging active levels of MMP-1 should decrease for an antiaging active, i.e., increased synthesis of dermal structural proteins together with reduced levels of the proteases that degrade them. For each analysis, the marker was identified in each of three sections (i.e., 3 sections/treatment/volunteer). Sections were optimally fixed. Routinely, following hydration in tris-buffered saline (TBS; 100 mM Tris, 150 mM NaCl), sections were solublised by addition of 0.5% Triton-X100 (10 minutes). Following washing, endogenous peroxidase activity was abolished by incubation with an excess of hydrogen peroxide in methanol (30 minutes). Sections were blocked prior to application of primary antibody (overnight incubation @4° C.). Negative controls were concurrently incubated with either block alone or control serum. Following incubation, sections were stringently washed with TBS prior to application of an appropriate biotinylated secondary antibody. This was further conjugated to the enzyme horseradish peroxidase using a commercially available kit following the manufacturers instructions (ABC Elite System, Vector Laboratory, Peterborough UK). Antibody was localised using Vector SG® as chromogen (10 minute incubation)—washing in TBS quenched this reaction. Sections were counterstained using Nuclear Fast Red and finally dehydrated through serial alcohols, cleared and permanently mounted.

TABLE 1

| Marker | Host | Clone | Fixation | Dilution |
|---|---|---|---|---|
| Fibrillin-rich | Mouse | NeoMarkers; | 4% Para- | 1:100 |
| microfibrils | | 11C1.3 | fomaldehyde | |
| Pro-collagen I | Rat | Chemicon | 4% Para- | 1:1000 |
| | | International; | fomaldehyde | |
| | | M-58 | | |
| Matrix- | Mouse | Abcam, | 4% Para- | 1:100 |
| Metalloprotease I | | ab2461 | fomaldehyde | |

Sections were randomised, blinded and examined on a Nikon OPTIPHOT microscope (Tokyo, Japan). For assessment of ECM and cornified envelope components, the degree of immunostaining was assessed on a 5 point semi-quantitative scale where 0=no staining and 4=maximal staining. Four sections (including control) were examined per subject per site. The degree of immunostaining was scored for three high power fields per section, and the average score calculated for each site/test area. Differences in the distribution between the test sites, and after application of test substances for varying periods of time, were assessed for significance using the repeated measures analysis of variance test (ANOVA). To assess whether age or baseline values affected outcome measures, data was tested using paired Student's t-tests. Both models were tested using SPSS+ software (v11.5, SPSS Inc., Ill. USA) with significance taken at the 95 or at the 80% confidence level.

### Results

### Erythema

All volunteers tolerated the patch test protocol well. Furthermore, all-trans RA produced marked erythema at the site of application. Erythema was not observed using any of the novel formulations.

### Fibrillin-1

Appreciation of all-trans RA (the “gold” standard) produced deposition of fibrillin-1proximal to the DEJ in the volunteers. Application of 0.2% PS-SLC resulted in significantly increased fibrillin-1 deposition in the exact same volunteers using the 95% confidence level. Application of the 80% confidence level also showed significant induction of fibrillin expression by 0.05% PS-SLC and 0.2% PS.

TABLE 2
Effect of novel formulation on fibrillin expression proximal to the DEJ.
Descriptive Statistics: procollagen I IHC

| | | | | | Std. |
|---|---|---|---|---|---|
| | N | Minimum | Maximum | Mean | Deviation |
| Vehicle | 5 | 0.00 | 1.83 | 1.2778 | 0.74536 |
| 0.2% PS-SLC | 5 | 1.33 | 3.22 | 2.267* | 0.83157 |
| 0.05% PS-SLC | 5 | 1.56 | 2.17 | 1.7778** | 0.23895 |
| 0.2% PS | 5 | 1.22 | 2.67 | 2.0000** | 0.64310 |
| 0.025% RA | 5 | 2.14 | 3.56 | 2.8625** | 0.53309 |
| Valid N (listwise) | 5 | | | | |

*p < 0.05,
**p < 0.2

Pro-Collagen I (pCI)

Application of all-trans RA had little effect on the deposition of pCI proximal to the DEJ following 4-day occluded application, in agreement with previous short-term studies, but it is known to increase levels in the longer term. However, 0.2% PS-SLC or 0.2% PS significantly increased pCI content in the papillary dermis using the 95 or the 80% confidence level, respectively.

TABLE 3
Effect of novel formulation on pro-collagen I
expression in the papillary dermis.
Descriptive Statistics: MMP1 IHC

| | | | | | Std. |
|---|---|---|---|---|---|
| | N | Minimum | Maximum | Mean | Deviation |
| Vehicle | 5 | 1.25 | 3.22 | 2.1389 | 0.82543 |
| 0.2% PS-SLC | 5 | 2.11 | 3.56 | 2.7790* | 0.52180 |
| 0.05% PS-SLC | 5 | 1.89 | 2.78 | 2.2472 | 0.33570 |
| 0.2% PS | 5 | 2.11 | 3.22 | 2.5556** | 0.42310 |
| 0.025% RA | 5 | 1.56 | 2.89 | 2.2667 | 0.59701 |
| Valid N (listwise) | 5 | | | | |

*p < 0.05,
**p < 0.2

### Matrix-Metalloprotease-1

Application of all-trans RA had no effect on the expression of MMP-1 following 4-day occluded application, in agreement with previous short-term studies. However, for this marker, both 0.05 and 0.2% concentrations of salicyloyl-phytosphingosine decreased MMP-1 levels significantly using the 95% confidence level. Application of phytosphingosine had no effect on MMP-1 expression.

TABLE 4
Effect of novel formulation on MMP-1 expression.
Descriptive Statistics: MMP-1 IHC

| | | | | | Std. |
|---|---|---|---|---|---|
| | N | Minimum | Maximum | Mean | Deviation |
| Vehicle | 5 | 11.33 | 43.178 | 33.95 | 13.01 |
| 0.2% PS-SLC | 5 | 13.67 | 31.33 | 22.72* | 10.47 |
| 0.05% PS-SLC | 5 | 0.00 | 37.33 | 18.40* | 13.33 |
| 0.2% PS | 5 | 13.17 | 46.33 | 33.23 | 15.10 |
| 0.025% RA | 5 | 11.54 | 62.75 | 36.82 | 16.01 |
| Valid N (listwise) | 5 | | | | |

*p < 0.05

From this study, it was shown that that topical occluded application of PS-SLC or PS restores the fibrillin-rich microfibrillar network proximal to the dermal-epidermal junction of photoaged skin, In addition, PS-SLC and PS also shows beneficial effects on procollagen I in the papillary dermis. Additionally, application of salicyloyl-phytosphingosine decreased MMP-1 levels significantly.

All of these effects will be useful for treating photodamaged as well as cellulitic skin conditions. These are the first ever results demonstrating the effects of these molecules on increasing dermal and dermoepidermal junctional molecules in skin. Sphingolipids have only been shown in the past to treat the barrier properties and the differentiation process of the skin.

### Example 2

The vehicle will usually form from 80 to 99.99%, preferably from 90 to 99.99% and most preferably from 98 to 99.98% by weight of the composition, and can, in the absence of other cosmetic adjuncts, form the balance of the composition.

The skin care formulation can be an aqueous solution, a water-in-oil (w/o) emulsion, an oil-in-water (o/w) emulsion, a dispersion of lipids, an aqueous, water-alcohol, oil or oil-alcohol gel, a solid stick, a wet-wipe or an aerosol.

If the vehicle itself is an (w/o) or (o/w) emulsion, it contains 5 to 50% of an oil phase and 47 to 94.95% water, with respect to the weight of the whole formulation,

The following table (i.e., Table 5) shows an example of a typical o/w formulation:

TABLE 5

| | | | |
|---|---|---|---|
| A) | TEGO ® Care PS | Methyl Glucose | 3.0% |
| | | Sesquistearate | |
| | TEGO ® Alkanol 18 | Stearyl Alcohol | 1.5% |
| | TEGIN ® M | Glyceryl Stearate | 3.5% |
| | TEGOSOFT ® liquid | Cetearyl Ethylhexanoate | 9.2% |
| | TEGOSOFT ® CT | Caprylic/Capric | 10.0% |
| | | Triglyceride | |
| B) | Glycerin | | 5.0% |
| | Water | | 65.45% |
| C) | TEGO ® Carbomer 134 | Carbomer | 0.2% |
| | TEGOSOFT ® liquid | Cetearyl-ethylhexanoate | 0.8% |
| | Phytosphingosine or | | 0.2% |
| | Salicyloyl-Phytosphingosine | | |
| | NaOH (10%) | | 0.65% |
| | Perfume | | 0.5% |
| | Preservative | | q.s. |

Preparation of the formulation:

Phase A) and B) were heated up to 65° C. Phase B) was added to A). The mixture was homogenized (e.g., using an Ultra Turrax) and cooled down under slow stirring. At 60° C., phase C) was added, followed by a short homogenization step. NaOH and the Perfume were added at 35° to 40° C.

### Example 3

Six female subjects were selected for a clinical trial of anticellulite activity of salicyloyl-phytosphingosine by topical application.

All volunteers showed the same cellulite intensity in both thigh areas. The scale ranged from 0 to 4, being 0=No cellulite; 1=Small bumps or depressions; 2=Striations and bumps; 3=Pronounced lumpiness of the skin and striations; 4=All of the above plus hard sub-surface nodules. All tested volunteers had cellulite with grades 1 and 2. The subjects were divided in 2 groups of 3 individuals each.

Group 1 was instructed to apply a formulation described in Example 2 containing 0.2% salicyloyl-phytosphingosine on the right thigh and the vehicle formulation without salicyloyl-phytosphingosine in the left thigh. Group 2 was instructed to apply the formulations vice versa. The subjects were taught to carry out the application two times a day, at morning and at night for a period of 2 months. Afterwards the cellulite condition was evaluated by comparison of the thigh diameter, the clinical grading and the subjective improvement.

The diameter was reduced by 4% on the salicyloyl-phytosphingosine treated thigh in comparison with the vehicle treated thigh.

The clinical grading was reduced by 27% on the salicyloyl-phytosphingosine treated thigh in comparison with the vehicle treated thigh.

The subjective improvement was increased by 34% on the salicyloyl-phytosphingosine treated thigh in comparison with the vehicle treated thigh.

The results above show that the composition containing salicyloyl-phytosphingosine effectively ameliorated the cellulite condition.

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention is not limited to the exact forms and details described and illustrated, but fall within the scope of the appended claims.

What is claimed is:

1. A method of treating and/or preventing cellulitic skin comprising applying a cosmetic composition to skin, wherein said composition comprises:
a) at least one salicyloyl-sphingoid base or a derivative thereof; and
b) a dermatologically acceptable carrier,
wherein said composition increases dermal structural proteins (collagen and fibrillin) and reduces levels of MMP-1.

2. The method of claim 1 where the at least one salicyloyl-sphingoid base is salicyloyl-phytosphingosine.

3. The method of claim 1 where the at least one salicyloyl-sphingoid base is salicyloyl-sphingosine, salicyloyl-sphinganine or salicyloyl-6-hydroxysphinganine.

4. The method of claim 1 where one or more of the hydroxyl groups of the sphingoid base-moiety of the at least one salicyloyl-sphingoid base is modified as organic and inorganic esters.

5. The method of claim 1 where one or more of the hydroxyl groups of the sphingoid base-moiety of the at least one salicyloyl-sphingoid base is modified by glycosylation.

6. The method of claim 1 where the composition comprising 0.001 to 20% by weight of the composition of said at least one salicyloyl-sphingoid base.

7. The method of claim 1 where the composition comprising 0.01 to 10% by weight of the composition of said at least salicyloyl-sphingoid base.

8. The method of claim 1 where the composition comprising 0.02 to 2% by weight of the composition of said at least salicyloyl-sphingoid base.

9. The method according to claim 1 where the composition further comprising at least one compound selected from the group consisting of
a) Sphingoid and phospholipids derivatives,
b) Antioxidants and vitamins,
c) Antiinflammatories,
d) Botanical agents,
e) Moisturising agents,
f) Skin whitening agents,
g) Peptides, modified peptides, protein hydrolysates,
h) Caffeine, and
i) Sunscreens and UV-Absorbers.
